(19) United States
(12) Patent Application Publication
(10) Pub. No.: US 2003/0012748 A1
(43) Pub. Date: Jan. 16, 2003

(54) Artificial fingernail composition

(76) Inventor: Douglas D. Schoon, Laguna Nigel, CA (US)

Correspondence Address: BROWN, MARTIN, HALLER & MCCLAIN LLP, 1660 UNION STREET, SAN DIEGO, CA, 92101-2926, US

(21) Appl. No.: 09/773,223

(22) Filed: Jan. 31, 2001

(51) Int. Cl. A61K 7/04

(52) U.S. Cl. 424/61

(57) ABSTRACT

The invention described herein is a composition used in the formation of artificial fingernail enhancements, which can be either full nail coatings or nail “tips”, and containing boron nitride as the primary pigment for coloration. While the use of boron nitride in various cosmetic formulations has been known, it has never before been used as a whitening pigment in artificial fingernail formulations in the manner described herein and the unique and beneficial properties of the addition of boron nitride in such compositions have heretofore been unknown and unrecognized.

### FIELD OF THE INVENTION

This invention generally relates to the compositions and methods for the formation of artificial human fingernails. More specifically, it relates to such compositions containing boron nitride (BN).

### BACKGROUND OF THE INVENTION

The application and maintenance of artificial nail enhancements by cosmetologists and nail arts technicians has become commonplace. An ever increasing number of clients patronize salons and boutiques specializing in the application of artificial nail enhancements, some seeking the latest nail fashion trends, others looking to improve on the appearance of their own weak, brittle or even malformed fingernails. The demand for increasingly durable, more attractive, easier to apply artificial nail enhancement formulations is a constant challenge for the industry.

Artificial nail enhancements are divided by product type and include: light-cure gels, liquid-and-powder or two-part systems, wraps and no-light gels. While the method of application and chemistry behind the various product types differ slightly, all product types facilitate the formation of the nail enhancement through the application of a thin coating of a viscous polymerizable material onto the surface of the natural nail, initially shaping the material and then allowing it to polymerize to hardness. Polymerization can be facilitated through the use of initiators or accelerants incorporated into the viscous formulation. Once the desired hardness has been achieved, the material coating the nail can be finaly shaped by filing, and then polished to present an attractive and natural appearance.

To date, the liquid-and-powder or two-part system constitutes the most widely used type of artificial nail enhancement in the world. The industry-wide standard for formulations used in two-part “liquid-and-powder” systems comprises a liquid binder which is a complex mixture of methacrylate monomers and a polymeric powder generally comprising monopolymeric ethyl methacrylate or copolymeric ethyl methacrylate-methyl methacrylate in varying ratios. Additionally, the polymeric powder acts as a carrier for polymerization initiators such as benzoyl peroxide, pigments for coloration and flow modifiers which are coated on the outside of the polymer resin bead.

The creation and application of an artificial nail enhancements onto a human fingernail is a precise art that takes time and practice to perfect. To form the viscous material that is applied directly onto the surface of the fingernail, a brush or similar tool is dampened in a reservoir or “dappen dish” containing the liquid binder, then is transferred to a reservoir containing the polymeric powder. The wetted brush is allowed to contact the polymeric powder is such a way as to form a dough-like “bead” at the end of the brush. The “bead” is transferred to the surface of the fingernail where it is shaped with the brush to form the desired coating. Curing time of the formed coating depends on the ratios of liquid binder, polymerization initiators and accelerators and polymeric powder used in the formulation. Typically, curing time is about from 60 seconds to 240 seconds from the time of initial mixing of the liquid and powder.

An important consideration in the formulation of artificial fingernail compositions is the consistency of the viscous “bead” that is formed on the nail technician's brush. The ideal consistency is that which can be easily moved and shaped with the brush, yet does not slump, sag or run. The key to achieving such working properties relies on the correct matching of liquid binder components with polymeric powder components. The liquid binder must display adequate solvency properties in order to dissolve, or properly dampen the polymeric powder. Conversely, the polymeric powder must be such that it is soluble in the liquid binder in order to achieve the desired consistency of the viscous “bead”.

When used as a thin film coating the fingernails, many artificial nail enhancement formulations polymerize in the presence of heat or light. The speed of polymerization is an important factor in the determination of whether an artificial nail formulation can be used successfully. The use of excess accelerates or catalysts may hasten polymerization but their addition can sometimes result in the finished nail composition being unacceptably brittle or prone to discoloration. On the other hand, to overly prolong the rate of polymerization can result in a fingernail application that fails to adequately harden or cures unevenly, having regions within the applied fingernail coating that only partially polymerize. In such cases, the nail technician must remove the entire fingernail enhancement and start the application process over with a completely new coating of the enhancement material.

Provided that the nail technician has managed to find the optimal combination of both the liquid binder and polymeric powder and has attained competency in the art of artificial fingernail enhancement application, there are still a number of problematic characteristics that are accepted as inherent in artificial fingernail compositions of the prior art. Because the artificial nail enhancement is applied directly to, and adheres to, the natural fingernail, normal growth of the natural fingernail results in obvious lines of demarcation where the application of the artificial fingernail is terminated (at the edge of the cuticle) and the new fingernail growth. Normal fingernail growth is the primary reason that regular maintenance or rebalancing is required for artificial fingernails. When a new coating of the artificial fingernail composition is applied over an existing coating, in order to “fill” the “gap” resulting from the out-growth of the natural fingernail, an obvious area of demarcation between the existing coating and the newly applied coating remains, even upon complete polymerization, and must consequently be covered using an opaque fingernail polish. Similarly, with pigmented artificial fingernail compositions, such as white and pink, if during the application of the composition to the natural fingernails the nail technician finds that the freshly applied “bead” of un-polymerized material is not sufficient to cover the entire surface of the natural fingernail, and additional material is required, “swirling” or “shadowing” marks remain where the two separately applied materials have been layered together.

Color stability and workability of the artificial fingernail enhancement both during the initial application of the material and after polymerization are considered major issues of importance to nail technicians using the two part “liquid-and-powder” system. If the nail technician uses a ratio of liquid to powder that is incorrect, giving a “bead” consistency that is either too moist or too dry, the performance of the enhancement will be dramatically affected. When the proper consistency is used, the nail enhancement will contain the correct amount of polymer powder, between 33-40% polymer. Too little polymer powder means less reinforcement and lower strength for the enhancement.

Titanium dioxide is well known in the cosmetic arts as providing absorptive and lamellar characteristics to a variety of cosmetic products. In the nail arts, specifically in service products such as artificial nail enhancements, titanium dioxide is used as a coloring agent, imparting a coloration that mimics the color of the nail plate of a natural fingernail. Higher percentages of titanium dioxide can be found in “white-tip” polymers which are used to create artificial nail extensions beyond the free edge of the natural fingernail and in the creation of a “French manicure”. While titanium dioxide has the ability to impart a high degree of whiteness to the nail enhancement, increased amounts of the pigment in artificial fingernail compositions adversely effects the workability of the product by causing the material to be too dry and prone to caking which makes the application process more difficult. Additionally, titanium dioxide displays a weak affinity for the polymer resin bead that acts as a carrier for the pigment. In artificial fingernail formulations containing higher amounts of titanium dioxide, the pigment falls off the resin bead and contaminates the monomer liquid resulting in a cloudy solution leaving the artificial nail enhancements with an undesirable milky appearance.

It would be desirable to provide an artificial fingernail composition that allows for the fabrication of a naturally white and bright artificial nail enhancement having reduced or eliminated amounts of titanium dioxide as well as providing an artificial fingernail composition that exhibits improved workability and color retention over the existing prior art.

### SUMMARY OF THE INVENTION

It is an advantage of the present invention to provide an artificial fingernail formulation that has improved workability over the existing art. As used herein, workability may be defined as the application of the artificial fingernail composition to the natural fingernail where it relates to powder “bead” control, powder “bead” consistency, quality of blending and surface coverage and ease with which the artificial nail enhancement may be shaped and filed.

It is a further advantage of the present invention to provide an artificial fingernail formulation that exhibits improved polymer bead adhesion.

Another advantage of the present invention is to provide an artificial fingernail formulation that retains a desirable white color without fading.

In an exemplary embodiment, boron nitride (BN) is added to an artificial fingernail formulation containing any combination of poly (ethyl methacrylate/methyl methacrylate), poly ethyl methacrylate, titanium dioxide and benzoil peroxide. The usable concentration of boron nitride in the artificial fingernail composition is between 0.1-10% with a preferred concentration between 1.0-3.0%.

In a further exemplary embodiment, boron nitride (BN) is added to an artificial fingernail formulation containing any combination of poly (ethyl methacrylate/methyl methacrylate), poly ethyl methacrylate and benzoil peroxide. The usable concentration of boron nitride in the artificial fingernail composition is between 0.1-10% with a preferred concentration between 1.0-3.0%.

In yet another exemplary embodiment, boron nitride (BN) is added to the monomer liquid of a two part liquid-powder artificial fingernail formulation to create a white liquid useful for creating nail enhancement “tips”. The addition of BN in this manner improves workability of the artificial fingernail enhancement and provides a product for creating white nail tips that exhibit no detectable ghosting or shadowing.

The artificial fingernail compositions of the present invention provides for a nail enhancement product that has a denser and more substantial feel and displays better manipulative characteristics when applied to the surface of a natural fingernail. During application, polymer powder formulated with boron nitride absorbs the monomer liquid more evenly enabling the nail technician to attain proper “bead” consistency which is vital to the performance of the artificial nail enhancement. A slightly slower rate of polymerization ensures that the enhancement will not become brittle with use and provides the nail technician with ample time to perfect and shape the enhancement. The addition of boron nitride imparts a bright, clean appearance to the finished nail enhancement, with a glossy shine and white color that does not fade over time. Additionally, boron nitride exhibits far greater powder bead adhesion than that observed for traditionally used whitening pigments such as titanium dioxide.

### DETAILED DESCRIPTION OF PREFERRED EMBODIMENTS

The invention described herein is a composition used in the formation of artificial fingernail enhancements, which can be either full nail coatings or nail “tips”, and containing boron nitride as the primary pigment for coloration. While the use of boron nitride in various cosmetic formulations has been known, it has never before been used as a whitening pigment in artificial fingernail formulations in the manner described herein and the unique and beneficial properties of the addition of boron nitride in such compositions have heretofore been unknown and unrecognized.

As previously noted, the widely used two-part system for the formation of artificial nail enhancements utilizes a liquid binder which is a complex mixture of methacrylate monomers and a dry polymeric powder generally comprising monopolymeric ethyl methacrylate and copolymeric ethyl methacrylate-methyl methacrylate in varying ratios, the preferred ratio generally being 50:50 monopolymeric ethyl methacrylate to copolymeric ethyl methacrylate-methyl methacrylate. The polymeric powder also acts to provide polymerization initiators and accelerants, flow modifiers and coloring agents to the composition, such components being conventional and well known in the art.

In the composition of the present invention, boron nitride is combined with or is substituted for titanium dioxide or other known whitening pigments as coloring agent. It has been discovered that the addition of boron nitride, in concentration ranges of 0.1-10% with a preferred concentration range of 1.0-3.0%, to known liquid-and-powder artificial fingernail compositions imparts an improved workability to the material. Compositions containing boron nitride exhibit decreased incidence of free edge wear and the polymerized product is more easily shaped and filed due to the internal lubricating characteristics of boron nitride on the powder bead.

Artificial nail enhancements formed from the composition of the present invention have been found to display enhanced whiteness and a diminished tendency towards fading over nail enhancements formed from prior art compositions. Similarly, artificial nail enhancements formed from the new compositions display superior blending capabilities over prior art compositions, showing no visible lines of demarcation, swirling or shadowing upon the addition of fresh material to previously applied material. Consequently, artificial nail enhancements created using the boron nitride composition of the present invention can be worn for longer periods of time before rebalancing of the enhancement is needed.

An important element of the present invention and specifically of boron nitride is the pigment's superior powder-bead adhesivity or adhesive characteristics over that of titanium dioxide. Because titanium dioxide adheres only weakly to the polymeric powder bead, the use of titanium dioxide in amounts greater than approximately 0.4% results in the contamination of the monomer liquid binder solution being used in the construction of the artificial nail enhancements. If use of the contaminated binder solution is continued, the resulting nail enhancements will be milky and unnatural in appearance. However, if less titanium dioxide is used, to avoid contamination of the liquid binder solution, the artificial nail enhancement will not have the white pigmentation considered desirable for the product. The superior powder-bead adhesivity of boron nitride allows for the addition of boron nitride to artificial fingernail formulations in much higher concentrations without causing the contamination of the liquid binder solution seen with titanium dioxide pigment while still imparting a bright, white color to the finished nail enhancement.

It will be evident from the above that there are other embodiments of this invention which those skilled in the art will recognize to be clearly within the scope and spirit of the invention even though not expressly stated above. The above detailed description is therefore intended to be exemplary only and the full scope of the invention is to be limited solely by the appended claims.

What is claimed is:

1. An artificial fingernail composition comprising a liquid binder solution and a dry polymeric powder, and further comprising:
boron nitride, or boron nitride in combination with titanium dioxide in quantities such that combination of the liquid binder solution, and the dry polymeric powder having the added boron nitride or the boron nitride in combination with the titanium dioxide results in an artificial fingernail enhancement with improved workability, powder bead adhesion, and color retention over the use of titanium dioxide alone.

2. The artificial fingernail composition of claim 1, wherein the liquid binder solution is any mixture of methacrylate monomers.

3. The artificial fingernail composition of claim 1, wherein the dry polymeric powder comprises any combination of monopolymeric ethyl methacrylate and copolymeric ethyl methacrylate-methyl methacrylate.

4. The artificial fingernail composition of claim 3, wherein the dry polymeric powder additionally comprises additives selected from a group comprising polymerization initiators, polymerization accelerants and flow modifiers, singly or in combination.

5. The artificial fingernail composition of claim 1, wherein the added boron nitride is in a concentration range of 0.1 to 10% with a preferred concentration range of 1.0 to 3.0%.

6. The artificial fingernail composition of claim 5, wherein the added boron nitride in a concentration range of 0.1 to 10% with a preferred concentration range of 1.0 to 3.0% displays enhanced powder-bead adhesivity.

7. The artificial fingernail composition of claim 5, wherein the added boron nitride in a concentration range of 0.1 to 10% with a preferred concentration range of 1.0 to 3.0% imparts improved workability to the resulting artificial fingernail enhancement.

8. The artificial fingernail composition of claim 7, wherein the improved workability to the resulting artificial fingernail enhancement comprises ease of application of the artificial fingernail enhancement and a decreased incidence of free edge wear of the resulting artificial fingernail enhancement.

9. The artificial fingernail composition of claim 1, wherein the added boron nitride has internal lubricating characteristics.

10. The artificial fingernail composition of claim 9, wherein the boron nitride having internal lubricating characteristics facilitates ease of filing and shaping of the resulting artificial fingernail enhancement.

11. The artificial fingernail composition of claim 1, wherein the added boron nitride imparts improved blending of the composition.

12. The artificial fingernail composition of claim 1, wherein the added boron nitride enhances whiteness of the resulting artificial nail enhancement.
